(19) United States
(12) Patent Application Publication
(10) Pub. No.: US 2011/0224626 A1
(43) Pub. Date: Sep. 15, 2011

(54) SAFETY NEEDLE CAP

(76) Inventors: Ming-Tien Lin, Nantou City (TW); Hsi-Hsiung Teng, Kaohsiung (TW)

(21) Appl. No.: 12/719,946

(22) Filed: Mar. 9, 2010

(51) Int. Cl. A61M 5/00

(52) U.S. Cl. 604/263

(57) ABSTRACT

A safety needle cap has a body and at least one holding segment. The body is tubular and has a closed end, an open end, a chamber and a bending segment. The chamber is defined in the body between the closed end and the open end to contain a needle. The bending segment is formed on an external surface of the body to make the body bendable. The at least one holding segment is formed on the body and each of the at least one holding segment has an extending stem and a pulling tab. The extending stem is formed on the body and has a free end opposite to the body and connected to the pulling tab. In use, the extending stem can prevent a tip of the needle from injuring a hand of a user when the needle is removed from or replaced in the body of the safety needle cap.

### BACKGROUND OF THE INVENTION

1. Field of the Invention

The present invention relates to a needle cap, and more particularly to a safety needle cap that can be operated and recovered safely and can prevent a used needles from being reused.

2. Description of Related Art

Needles can be classified according use, syringe needles, intravenous needles, scalp needles or blood drawing needles. A needle cap is mounted around a tip of the needle to prevent accidental contamination of the needle during transportation, handling or after use. In use, the needle is held by one hand and the needle cap is removed by the other hand.

Although the conventional needle cap can provide a covering effect for the needle, the tip of the needle may injure the hand holding the conventional needle cap, especially if the hand is accidentally jogged or knocked on removal or during replacement. Furthermore, a used needles may be reused accidentally so risking disease transmission.

In order to improve the safety of using the needle cap, a safety needle cap is proposed that can be removed and replaced safely while also preventing reuse of a needle to overcome the aforementioned shortcomings.

### SUMMARY OF THE INVENTION

The main objective of the invention is to provide a safety needle cap that can be removed and replaced safely and prevents reuse of a needle.

The needle cap in accordance with the present invention has a body and at least one holding segment. The body is tubular and has an external surface, a closed end, an open end, a chamber and a diameter. The chamber is defined in the body between the closed end and the open end to contain a needle. The at least one holding segment is formed on the body and the at least one holding segment has an extending stem. The extending stem is formed on the body and has a free end opposite to the body. In use, one hand holds the extending stem of the at least one holding segment and the other hand holds the needle, and the extending stem can prevent a tip of the needle from injuring either hand when the needle is removed from or replaced in the body of the safety needle cap.

Other objects, advantages and novel features of the invention will become more apparent from the following detailed description when taken in conjunction with the accompanying drawings.

### BRIEF DESCRIPTION OF THE DRAWINGS

FIG. 1 is a perspective view of a first embodiment of a safety needle cap in accordance with the present invention mounted around a syringe needle;

FIG. 2 is an exploded perspective view of a second embodiment of a safety needle cap in accordance with the present invention with an intravenous needle;

FIG. 3 is an enlarged side view in partial section of the safety needle cap in FIG. 2 mounted around the intravenous needle;

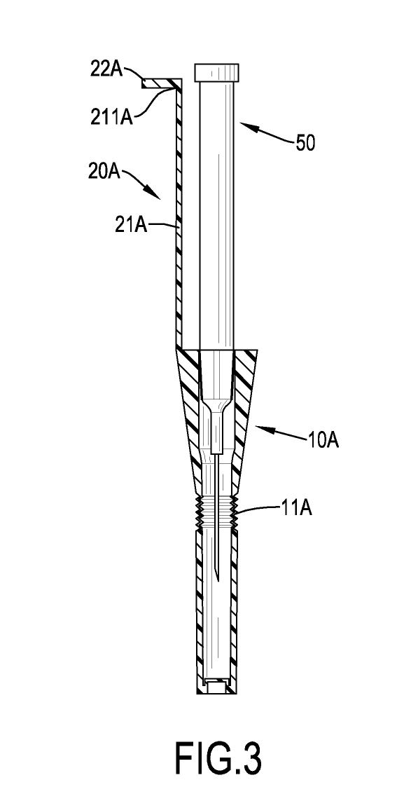

FIG. 4 is an operational side view in partial section of the safety needle cap in FIG. 3;

FIG. 5 is a perspective view of a third embodiment of a safety needle cap in accordance with the present invention;

FIG. 6 is an exploded perspective view of a fourth embodiment of a safety needle cap in accordance with the present invention with a scalp needle;

FIG. 7 is a perspective view of a fifth embodiment of a safety needle cap in accordance with the present invention mounted around a syringe needle;

FIG. 8 is an operational side view of the safety needle cap in FIG. 7;

FIG. 9 is a side view in partial section of a sixth embodiment of a safety needle cap in accordance with the present invention mounted around a large size syringe needle; and

FIG. 10 is a side view in partial section of a seventh embodiment of a safety needle cap in accordance with the present invention mounted around a large size syringe needle.

### DETAILED DESCRIPTION OF PREFERRED EMBODIMENT

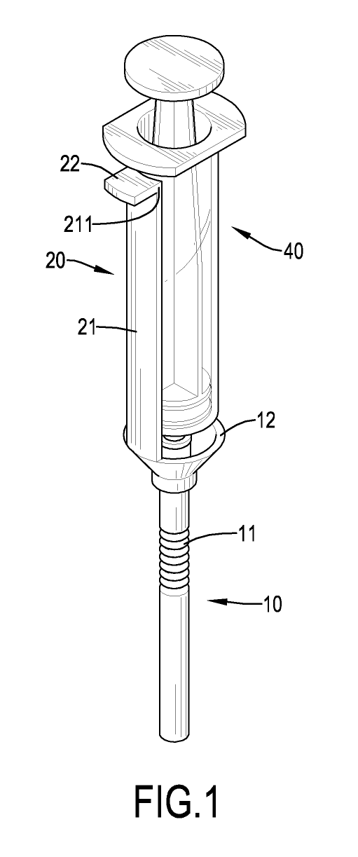

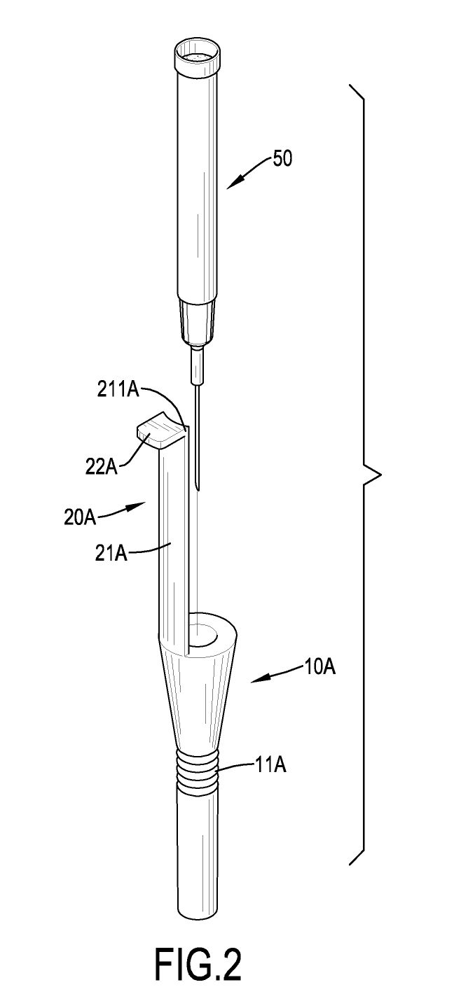

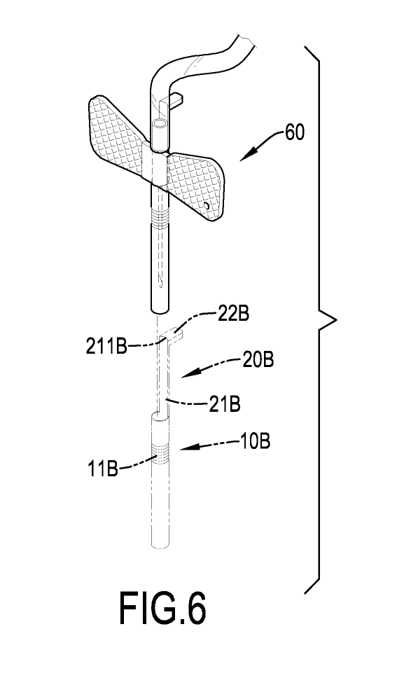

With reference to FIGS. 1, 2, 5 to 7, 9 and 10, a safety needle cap in accordance with the present invention comprises a body (10, 10A, 10B, 10C, 10D, 10E) and at least one holding segment (20, 20A, 20B, 20C, 20D, 20E).

The body (10, 10A, 10B, 10C, 10D, 10E) is tubular, is mounted around a syringe needle (40), an intravenous needle (50), a scalp needle (60) or a large size syringe needle (70) as shown in FIGS. 1, 2, 6, 7, 9 and 10, has an external surface, a closed end, an open end, a chamber, a diameter and a bending segment (11, 11A, 11B, 11C) and may have a top cover (12, 12C). The chamber is defined in the body (10, 10A, 10B, 10C, 10D, 10E) between the closed end and the open end to contain the needle (40, 50, 60, 70).

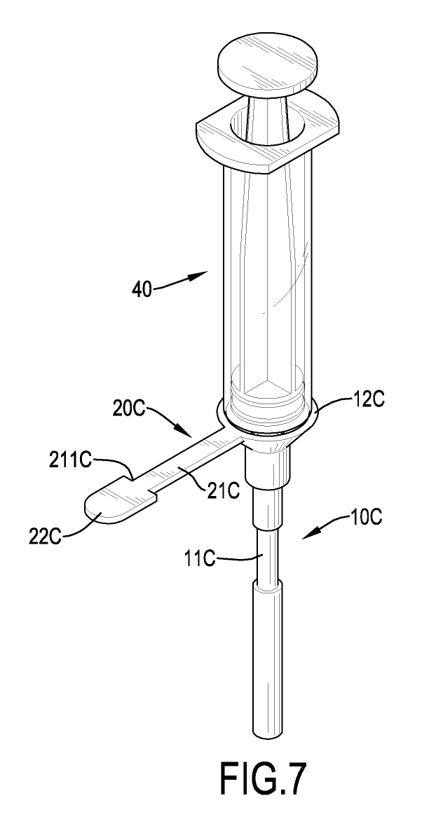

The bending segment (11, 11A, 11B, 11C) is formed on the external surface of the body (10, 10A, 10B, 10C) to make the body (10, 10A, 10B, 10C) bendable. Preferably, with reference to FIGS. 1, 2, 5 and 6, the bending segment (11, 11A, 11B) is formed on the external surface of the body (10, 10A, 10B) near the open end and is a shrinking structure. Preferably, with reference to FIG. 7, the bending segment (11C) is formed on the external surface of the body (10C) near the open end and has a diameter smaller than the diameter of the body (10C) to form a bending structure.

With reference to FIGS. 1, 7 and 9, the top cover (12, 12C) is funnel shaped and is formed on and protrudes from the open end of the body (10, 10C).

The at least one holding segment (20, 20A, 20B, 20C, 20D, 20E) is formed on the body (10, 10A, 10B, 10C, 10D, 10E) and each one of the at least one holding segment (20, 20A, 20B, 20C, 20D, 20E) has an extending stem (21, 21A, 21B, 21C, 21D, 21E) and a pulling tab (22, 22A, 22B, 22C, 22D, 22E).

With reference to FIGS. 1, 2, 5 to 7 and 9, the extending stem (21, 21A, 21B, 21C, 21D) is formed on the open end of the body (10, 10A, 10B, 10C, 10D) and has a free end (211, 211A, 211B, 211C, 211D) opposite to the open end of the body (10, 10A, 10B, 10C, 10D). The extending stem (21, 21A, 21B, 21D) is longitudinally formed on and protrudes from the open end of the body (10, 10A, 10B, 10D) as shown in FIGS. 1, 2, 6 and 9 or the extending stem (21C) is transversally formed on and protrudes from the open end of the body (10C) as shown in FIG. 7. With reference to FIGS. 1 and 7, the extending stem (21) is longitudinally formed on the top cover (12) of the body (10) or the extending stem (21C) and is transversally formed on the top cover (12C) of the body (10C).

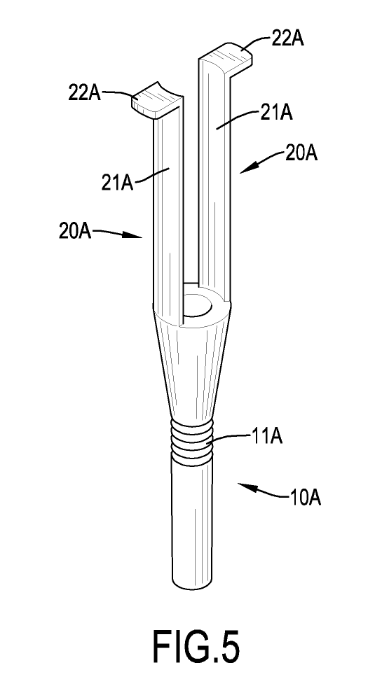

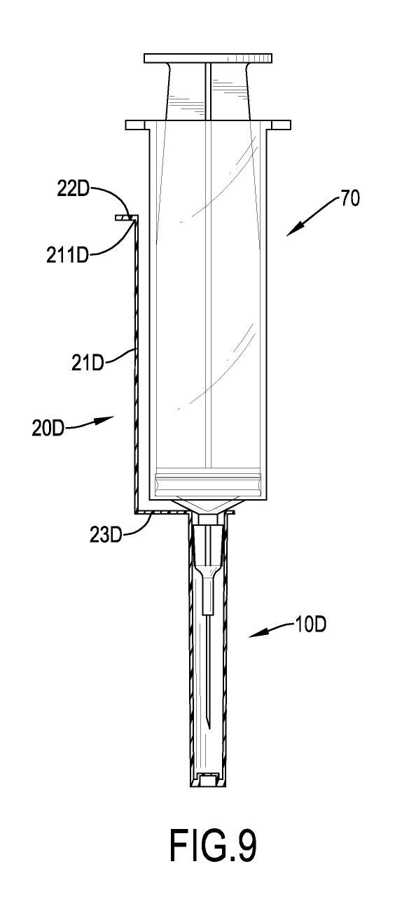

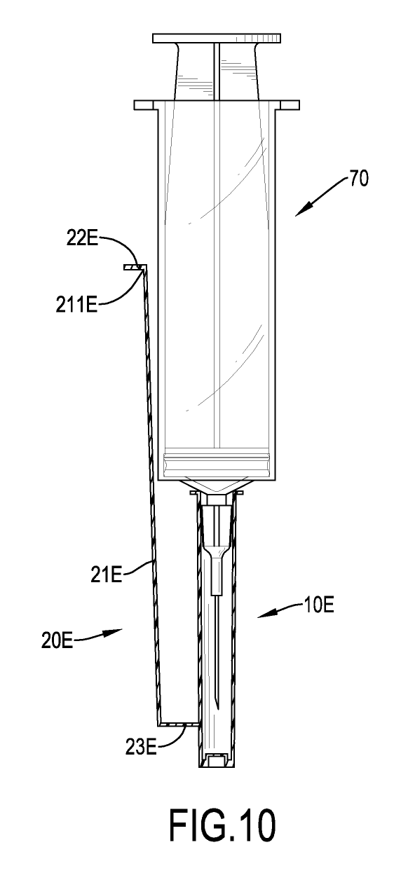

Preferably, with reference to FIG. 5, the safety needle cap has two holding segments (20A) longitudinally formed on and protruding from the open end of the body (10A). With reference to FIG. 10, the extending stem (21E) is formed on the external surface of the body (10E) between the open end and the closed end and has a free end (211E). Preferably, with further reference to FIGS. 9 and 10, the extending stem (21D, 21E) has a connecting panel (23D, 23E) transversally formed on the extending stem (21D, 21E) at an end opposite to the free end (211D, 211E) and connected to the body (10D, 10E) to prevent the extending stem (21D, 21E) from interfering with the large size syringe needle (70).

The pulling tab (22, 22A, 22B, 22C, 22D, 22E) is formed on the free end (211, 211A, 211B, 211C, 211D, 211E) of the extending stem (21, 21A, 21B, 21C, 21D, 21E) to allow the extending stem (21, 21A, 21B, 21C, 21D, 21E) to be pulled easily.

In use, with reference to FIGS. 1, 2, 6, 7, 9 and 10, the needle (40, 50, 60, 70) is removed from the safety needle cap in accordance with the present invention, one hand holds the pulling tab (22, 22A, 22B, 22C, 22D, 22E) of the holding segment (20, 20A, 20B, 20C, 20D, 20E) and the other hand pulls the needle (40, 50, 60, 70) out of the body (10, 10A, 10B, 10C, 10D, 10E). During removal, direct contact between the hand that holds the pulling tab (22, 22A, 22B, 22C, 22D, 22E) and the needle (40, 50, 60, 70) is prevented by the extending stem (21, 21A, 21B, 21C, 21D, 21E). In other words, the hand that holds the pulling tab (22, 22A, 22B, 22C, 22D, 22E) is kept away from the tip of the needle (40, 50, 60, 70). Then, the hand that holds the pulling tab (22, 22A, 22B, 22C, 22D, 22E) or the free end of the extending stem (21, 21A, 21B, 21C, 21D, 21E) is prevented from injury by the extending stem (21, 21A, 21B, 21C, 21D, 21E) even if either hand is accidentally knocked or jogged when removing or replacing the needle (40, 50, 60, 70).

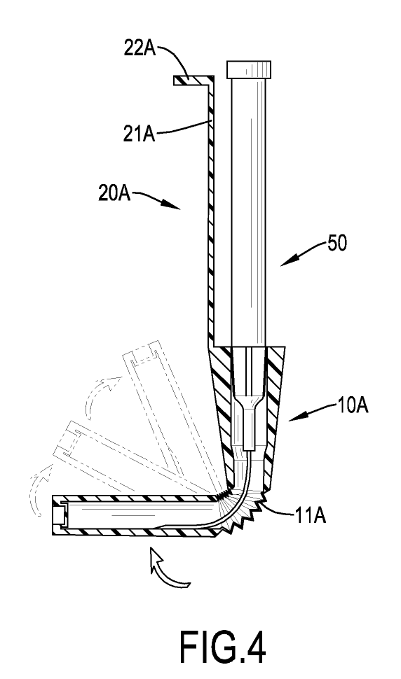

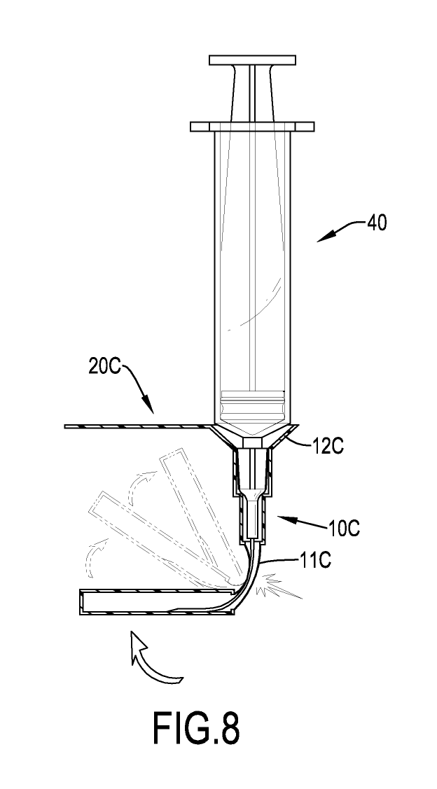

After use, the needle (40, 50, 60, 70) is replaced in the needle cap and the tip of the needle (40) is guided into the chamber of the body (10, 10A, 10B, 10C, 10D, 10E) by the extending stem (21, 21A, 21B, 21C, 21D, 21E) of the holding segment (20, 20A, 20B, 20C, 20D, 20E). Accordingly, the hand that holds the pulling tab (22, 22A, 22B, 22C, 22D, 22E) or the free end of the extending stem (21, 21A, 21B, 21C, 21D, 21E) is kept from contacting the tip of the needle (40, 50, 60, 70). After the used needle (40, 50, 60) is mounted in the chamber of the body (10, 10A, 10B, 10C), the body (10, 10A, 10B, 10C) can be bent as shown in FIGS. 4 and 8 by the bending segment (11, 11A, 11B, 11C) to bend the needle (40, 50, 60) in the chamber of the body (10, 10A, 10B, 10C). Thus, the used, bent needle (40, 50, 60) is prevented from reuse.

The safety needle cap in accordance with the present invention can prevent the hand that holds the pulling tab (22, 22A, 22B, 22C, 22D, 22E) or the free end of the extending stem (21, 21A, 21B, 21C, 21D, 21E) from being injured by the tip of the needle (40, 50, 60, 70) and the bending segment (11, 11A, 11B, 11C) of the body (10, 10A, 10B, 10C) can provide a bending effect to the needle (40, 50, 60) to prevent the used needles (40, 50, 60) from being reused.

Even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

1. A safety needle cap has a body being tubular and having an external surface, a closed end, an open end, a chamber defined in the body and a diameter, and at least one holding segment formed on the body and each one of the at least one holding segment having an extending stem formed on the body and having a free end opposite to the body, characterized in that the safety needle cap comprises:
the body having a bending segment being a shrinking structure and formed on the external surface of the body to make the body bendable.

2-4. (canceled)

5. The safety needle cap as claimed in claim 1, wherein each one of the at least one holding segment has a pulling tab formed on the free end of the extending stem to provide a pulling effect to the body.

6. The safety needle cap as claimed in claim 5, wherein the extending stem of each one of the at least one holding segment is longitudinally formed on and protrudes from the open end of the body.

7. The safety needle cap as claimed in claim 5, wherein the extending stem of each one of the at least one holding segment is transversally formed on and protrudes from the open end of the body.

8. The safety needle cap as claimed in claim 5, wherein the extending stem of each one of the at least one holding segment is formed on the external surface of the body between the open end and the closed end.

9. The safety needle cap as claimed in claim 5, wherein
the body has a funnel shaped top cover formed on and protruding from the open end of the body; and
the extending stem of each one of the at least one holding segment is longitudinally formed on the top cover of the body.

10. The safety needle cap as claimed in claim 5, wherein
the body has a funnel shaped top cover formed on and protruding from the open end of the body; and
the extending stem of each one of the at least one holding segment is transversally formed on the top cover of the body.

11. The safety needle cap as claimed in claim 6, wherein the safety needle cap has two holding segments longitudinally formed on and protruding from the open end of the body.

12-20. (canceled)

21. A safety needle cap has a body being tubular and having an external surface, a closed end, an open end, a chamber defined in the body and a diameter and at least one holding segment formed on the body and each one of the at least one holding segment having an extending stem formed on the body and having a free end opposite to the body, characterized in that the safety needle cap comprises
the body having a bending segment formed annularly on the external surface of the body and having a diameter smaller than the diameter of the body to form a step bending structure between the closed end and the open end of the body to face an outer surface of a needle.

22. The safety needle cap as claimed in claim 21, wherein each one of the at least one holding segment has a pulling tab formed on the free end of the extending stem to provide a pulling effect to the body.

23. The safety needle cap as claimed in claim 22, wherein the extending stem of each one of the at least one holding segment is longitudinally formed on and protrudes from the open end of the body.

24. The safety needle cap as claimed in claim 22, wherein the extending stem of each one of the at least one holding segment is transversally formed on and protrudes from the open end of the body.

25. The safety needle cap as claimed in claim 22, wherein the extending stem of each one of the at least one holding segment is formed on the external surface of the body between the open end and the closed end.

26. The safety needle cap as claimed in claim 22, wherein
the body has a funnel shaped top cover formed on and protruding from the open end of the body; and
the extending stem of each one of the at least one holding segment is longitudinally formed on the top cover of the body.

27. The safety needle cap as claimed in claim 22, wherein
the body has a funnel shaped top cover formed on and protruding from the open end of the body; and
the extending stem of each one of the at least one holding segment is transversally formed on the top cover of the body.

28. The safety needle cap as claimed in claim 22, wherein the safety needle cap has two holding segments longitudinally formed on and protruding from the open end of the body.
